(19) United States
(12) Patent Application Publication
(10) Pub. No.: US 2025/0273321 A1
(43) Pub. Date: Aug. 28, 2025

(54) PATIENT-SPECIFIC VISUALIZATION OF INFUSION COVERAGE USING SEGMENTED 3D BRAIN REPRESENTATIONS

(71) Applicant: ClearPoint Neuro, Inc., Solana Beach, CA (US)

(72) Inventors: Lyubomir ZAGORCHEV, Burlington, MA (US); Joseph BURNETT, Rancho Santa Fe, CA (US)

(21) Appl. No.: 18/584,583

(22) Filed: Feb. 22, 2024

(51) Int. Cl. G16H 30/40; A61B 5/00; G06T 7/12; G06T 7/62; G06T 17/20

(52) U.S. Cl. CPC G16H 30/40; A61B 5/742; G06T 7/12; G06T 7/62; G06T 17/20; G06T 2200/24; G06T 2207/30016; G06T 2210/21; G06T 2210/41

(57) ABSTRACT

Systems and methods provide automated systems for visualizing and estimating infusion coverage within a target brain region. Such accurate infusion coverage visualization and estimation enables intraoperative monitoring and adjustment of infusion parameters (e.g., cannula tip location, infusate delivery flow rate, etc.) for achieving optimal/improved infusion coverage for a given drug therapy. Accordingly, examples of the presently disclosed technology can improve the efficacy and safety of drug therapies delivered to the brain.

### TECHNICAL FIELD

The present disclosure relates generally to medical technologies, and more particularly, some examples relate to visualizing infusion coverage for drug therapies delivered to the brain.

### BACKGROUND

Drug therapies delivered to the brain can treat various genetic and acquired brain diseases. Delivering these therapies typically involves using a targeting cannula to administer/deliver infusate (i.e., drug) at one or more infusion points within a target brain region (i.e., a brain region targeted for therapy). Depending on the drug therapy and/or target brain region (e.g., tissue properties and local geometry of the target brain region), infusate coverage within the target brain region (i.e., the extent to, and manner in which, delivered infusate is volumetrically distributed within the target brain region) can be a crucial factor for optimizing/improving the efficacy and safety of the drug therapy. For example, a given drug therapy may be most effective when a particular volume of infusate is distributed uniformly across an entire target brain region.

Various infusion parameters may influence infusate coverage within a target brain region, including infusate flow rate (infusate flow rate may be controlled by an infusion pump which pushes infusate through the targeting cannula into the brain), targeting cannula tip/infusion point location, etc. For example, broader infusate coverage can be achieved by delivering infusate at multiple infusion points along a surgical trajectory within the target brain region. The delivered infusate may then flow/spread to larger volumes of the target brain region.

### BRIEF DESCRIPTION OF THE DRAWINGS

The present disclosure, in accordance with one or more various examples, is described in detail with reference to the following figures. The figures are provided for purposes of illustration only and merely depict examples.

FIG. 1 depicts an example shape-constrained deformable brain model, in accordance with various examples of the presently disclosed technology.

FIG. 2 depicts an example adaption of a shape-constrained deformable brain model to a scan of a patient's brain to generate a patient-specific 3D mesh brain representation, in accordance with examples of the presently disclosed technology.

FIG. 3 depicts an example patient-specific segmented 3D brain representation, in accordance with various examples of the presently disclosed technology.

FIG. 4 depicts an example flow diagram that may be used to visualize infusate delivered to a target brain region, in accordance with various examples of the presently disclosed technology.

FIG. 5 depicts an example patient-specific segmented 3D brain representation, in accordance with an example of the presently disclosed technology.

FIG. 6 depicts an example visualization of an infusate delivery representation of infusate delivered to a target brain structure/region, in accordance with various examples of the presently disclosed technology.

FIGS. 7-10 depict example visualizations of 3D infusate delivery representations combined with the 3D brain structure representation 500 of FIG. 5, in accordance with various examples of the presently disclosed technology.

FIG. 11 is an example computing component that may be used to implement various features of examples described in the present disclosure.

The figures are not exhaustive and do not limit the present disclosure to the precise form disclosed.

### DETAILED DESCRIPTION

As described above, infusate coverage within a target brain region can be a crucial factor for optimizing/improving the efficacy and safety of a drug therapy. Accordingly, real-time (as used herein “real-time” may refer to approximate “real-time” as well) visualization of infusion coverage within a target brain region would present a tremendous opportunity for optimizing/improving the efficacy and safety of drug therapies delivered to the brain. For example, such real-time visualization could enable intraoperative monitoring and adjustment of infusion parameters (e.g., cannula tip location, infusate delivery flow rate, etc.) to achieve optimal/improved infusion coverage for a given drug therapy.

Unfortunately, existing technologies are largely incapable of accurate, real-time estimation of infusion coverage within a target brain region. Accordingly, neurosurgeons typically rely on their own visual assessment of inter-operative images/scans (e.g., MRI scans or CT scans obtained during an infusion procedure) to gauge infusion coverage. Visual assessment generally requires the infusate be mixed with a contrast agent that is visible in the images/scans (e.g., Gadolinium). This manual approach can be subjective and highly inaccurate. In addition, current visualizations of infusate coverage (e.g., inter-operative MRI or CT scans) typically hide structural boundaries of brain structures (including a target brain region), significantly complicating visual assessment during an infusion. Further still, in operating rooms, or in the absence of inter-operative images/scans, visual assessment of infusion coverage is conventionally not performed because accurate real-time visuals are not conventionally available.

As described above, poor infusion coverage estimation can result in sub-optimal infusion coverage, which may lead to ineffective/unsafe drug therapies or failed clinical trials. Relatedly, because existing technologies struggle to estimate patient-specific infusion coverage in real-time, neurosurgeons typically rely on standardized (i.e., non-patient specific) clinical protocols to define infusion parameters for a given drug therapy. Because infusion parameters (e.g., infusion flow rates) can vary from person-to-person, brain region-to-brain region, procedure-to-procedure, etc.-these standardized clinical protocols can lead to sub-optimal infusion coverage for particular patients/procedures, which again may lead to ineffective and/or unsafe drug therapies.

Against this backdrop, examples of the presently disclosed technology provide automated systems and methods for accurately visualizing infusion coverage within a target brain region in real-time from interoperative images/scans during surgical procedures or from biophysical infusion modeling from a simulation. Such accurate and real-time infusion coverage estimation enables intraoperative monitoring and adjustment of infusion parameters (e.g., cannula tip location, infusate delivery flow rate, etc.) for achieving optimal/improved infusion coverage for a given drug therapy. Accordingly, examples of the presently disclosed technology can improve the efficacy and safety of drug therapies delivered to the brain.

Examples of the presently disclosed technology can automatically visualize infusion coverage in a target brain region by (1) segmenting a 3D brain structure representation of the patient's brain that includes a 3D brain sub-representation representing a target brain structure/region (i.e., referred to herein as a target 3D brain sub-representation) to define 3D boundaries of the target brain region; (2) based on the 3D boundaries, splitting a infusate delivery representation (sometimes referred to herein as a “representation of infusate”) to the target brain region into infusate within the target brain region (i.e., infusate which is located within the intended target brain region) and infusate outside the target brain region (i.e., infusate which is not staying within the intended target brain region); (3) mapping the split infusate delivery representation to the 3D target brain sub-representation according to a color table; (4) extracting 3D infusate delivery sub-representations for infusate within the target brain region (e.g., located inside or contained within the 3D boundaries) and for infusate outside the target brain region (e.g., located outside the 3D boundaries) from the mapped representations of infusate and generate a 3D visualization of the 3D infusate delivery sub-representations and the 3D target brain sub-representation; and (5) based on the 3D representations, estimating a level of coverage for delivered infusate within the target brain region.

Examples disclosed herein provide a neurosurgeon with visualized estimation of infusion coverage in real-time. For instance, examples may generate visualizations of infusate delivered inside of and outside of a target brain region using a mappings of contrasting colors. The mapping can comprise accessing a color lookup table that associates a unique visual attribute or indicator (e.g., colors or other visually distinguishing attributes) to infusate located within the target brain region and infusate located outside the target brain region. For example, the 3D infusate delivery sub-representation for infusate located within the target brain region may be visualized using a first visual indicator (e.g., green) and the 3D infusate delivery sub-representation for infusate outside the target brain region may be visualized using a second visual indicator (e.g., red). The 3D target brain sub-representation may be visualized using a third visual indicator (e.g., grey). Other visual indicators and colors may be used as desired. In this way, the disclosed technology can provide a clear visualization that neurosurgeons can use to readily assess the infusion coverage within a target brain region in real-time.

As will be described in greater detail below, the segmented 3D representation of the patient's brain may be a computerized 3D representation of the patient's brain comprised of individual 3D segments/sub-representations representing various regions/structures of the patient's brain (e.g., sub-cortical structures such as the putamen, thalamus, etc.). Accordingly, the segmented 3D representation of the patient's brain may identify—within the computerized 3D representation of the patient's brain—the various brain regions/structures of the patient, including the target brain region. Thus, by computing intersections between the 3D infusate delivery sub-representations and the segmented 3D target brain sub-representation, examples of the disclosed technology can generate a visualization of the infusate delivered to the target brain region. In certain examples, such computation may comprise combining/overlaying the 3D infusate delivery sub-representations and the 3D target brain sub-representation and detecting intersections therebetween. The visualization can provide a visual representation of infusate coverage metrics. Furthermore, by comparing the 3D infusate delivery sub-representations and the segmented 3D target brain sub-representation, examples of the disclosed technology can estimate infusate coverage metrics, such as a volume for delivered infusate within the target brain region. Accordingly, examples can estimate the volume for delivered infusate within the target brain region based on a volume of 3D infusate delivery representation for the infusate within the 3D target brain sub-representation. The estimated volume for the delivered infusate can be compared to a volume of the 3D target brain sub-representation to estimate a coverage percentage. Examples disclosed herein can also estimate a leakage volume (e.g., volume of infusate outside the target brain region) based on a volume of the 3D infusate delivery sub-representation for the infusate outside of the 3D target brain sub-representation. In various instances, the disclosed technology can measure the volumes of the 3D infusate delivery sub-representations and the 3D target brain sub-representation by counting a number of voxels of each respective 3D representation.

Utilizing techniques described above, examples can estimate infusion coverage within a target brain region and provide notifications to a neurosurgeon which visualize estimated infusion coverage in real-time. For instance, examples may visualize estimated infusion coverage using a “traffic light” notion: i.e., red (i.e., complete coverage achieved/stop infusion), yellow (i.e., nearly complete coverage achieved/continue infusion with caution), and green (i.e., incomplete infusion coverage/continue infusion). Here, infusion can be stopped when a certain percentage of coverage for the targeted brain region has been achieved.

As described above, by providing accurate, real-time infusion coverage estimations, examples of the presently disclosed technology can improve the efficacy and safety of drug therapies delivered to the brain in numerous ways. For instance, examples improve accuracy and responsiveness for inter-operative monitoring, which can allow a neurosurgeon to adjust infusion parameters more intelligently during an infusion procedure. Relatedly, examples may be used to change the current practice of setting up fixed clinical protocols for pharma trials. Optimal dosage of delivered infusate/therapeutic can and should be estimated during a pharmaceutical trail based on patient-specific data. Accordingly, infusion coverage estimates of the presently disclosed technology can reduce variability in current practice for pharmaceutical trials, thereby improving their safety and efficacy. As described above, examples can also estimate a leakage volume of delivered infusate located outside of a target brain region, can lead to unsafe outcomes. Accordingly, by visualizing/estimating a leakage volume of infusate in real-time, examples of the presently disclosed technology enable inter-operative adjustment of infusion parameters to reduce further infusate leaking.

Relatedly, examples disclosed herein may utilize biophysical infusion modeling to simulate drug therapies delivered to the brain. The biophysical infusion model can infusate propagation in a 3D representation of the patient's brain, including the 3D target brain sub-representation, and generate simulated infusate delivery data. The simulated infusate delivery data can be used to provide the representation of infusate delivered, which can be split based on the 3D boundaries of the target brain sub-structure sub-representation and used to a level of coverage, as well as leakage volume. The estimates can then be used to adjust infusion parameters intelligently prior to an infusion procedure, which can improve overall effectiveness of the therapy through optimize delivery.

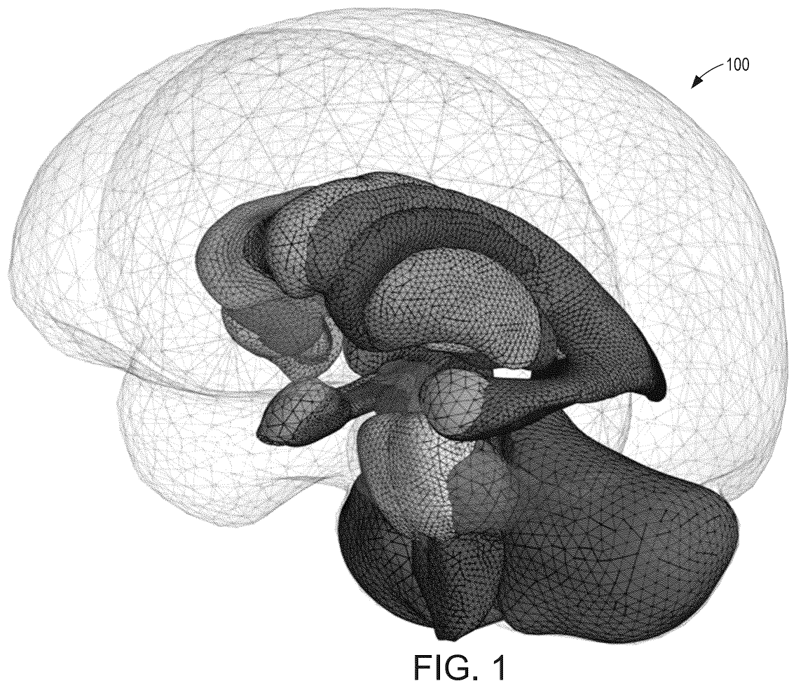

FIG. 1 depicts an example shape-constrained deformable brain model 100, in accordance with various examples of the presently disclosed technology. Shape-constrained deformable brain model 100 may be a computerized 3D mesh representation of a generalized human brain (i.e., a non-patient-specific 3D representation of the human scalp, skull, brain, etc.) that preserves mesh vertex-based correspondences during mesh adaption to patient-specific data/scans using shape-constrained deformation. Shape-constrained deformable brain model 100 can be derived as an average/mean mesh from a set of training data.

As depicted, shape-constrained deformable brain model 100 comprises mesh elements and mesh vertices at the junctions of adjoining/adjacent mesh elements. Each mesh element of shape-constrained deformable brain model 100 may represent a different brain region. In the example of FIG. 1, the mesh elements of shape-constrained deformable brain model 100 comprise triangles, but in other examples mesh elements may comprise different shapes.

In general, a mesh may refer to a representation of a larger domain (e.g., a volume or surface) comprised of smaller discrete cells called mesh elements (e.g., mesh triangles or other shapes), and mesh vertices at the junctions of adjacent/adjoining mesh elements. Meshes can be used to compute solutions to equations across individual mesh elements, which then can be used to approximate solutions over the larger domain.

As depicted (and as will be discussed below), shape-constrained deformable brain model 100 may comprise individual 3D segments/sub-representations representing various structures of the brain (e.g., the cortical surface, sub-cortical structures such as the globus pallidus, the putamen, the thalamus, etc.).

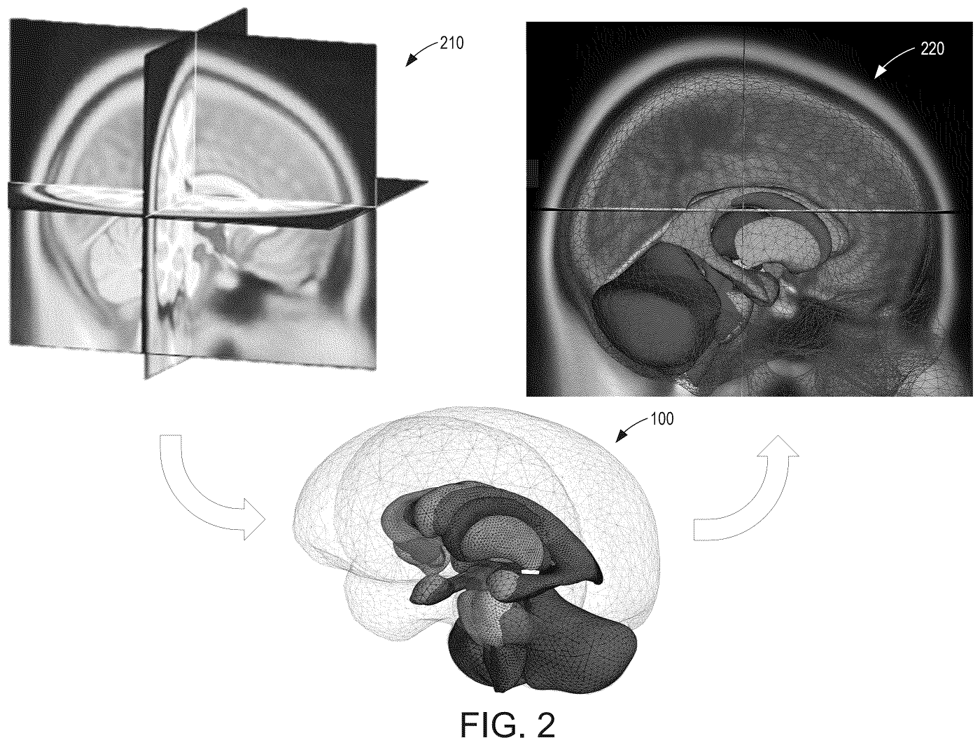

FIG. 2 depicts an example adaption of shape-constrained deformable brain model 100 generalized to one or more scans 210 of a patient's brain to generate a patient-specific 3D brain representation 220 and a dense deformation field that transforms a mean shape of shape-constrained deformable brain model 100 to the scan 210 of the patient's brain, in accordance with examples of the presently disclosed technology. The shape-constrained deformable brain model 100 can be adapted to structural scans, such as volumetric structural scans, of a patient's brain (e.g., MRI scans, CT scans, etc.) to generate a patient-specific 3D brain representation (also referred to herein as a patient-specific 3D brain structure representation). As depicted in FIG. 2, patient-specific 3D brain representation 220 may comprise individual 3D mesh sub-representations/segments representing individual brain structures of the patient (referred to herein as patient-specific 3D brain structure sub-representations). The patient-specific 3D brain structure sub-representations may represent the various brain structures (e.g., cortex structures, sub-cortical structures, etc.) of the patient's brain.

As alluded to above, shape-constrained deformable brain model 100 may be a computerized 3D mesh representation of a generalized human brain (e.g., a non-patient-specific 3D representation of the human brain) that preserves mesh vertex-based correspondences during mesh adaption to patient-specific data/scans using shape-constrained deformation. Namely, the process of mesh adaption generates a dense deformation field that transforms voxels from the coordinate space of the shape-constrained deformable brain model 100 (e.g., the shape-constrained deformable brain model 100 coordinate space) to a coordinate space of an imaging system (such as but not limited to Digital Imaging and Communications in Medicine (DICOM) standard interchange protocol) of scan(s) 210 (e.g., the scan(s) 210 coordinate space). The dense deformation field can leverage shape-constrained deformation to constrain deformation to an apriori derived mean mesh (e.g., shape-constrained deformable brain model 100). The dense deformation field can use a penalty term estimated from the mean mesh (e.g., estimated from shape-constrained deformable brain model 100) that prevents topological changes during mesh adaptation, which may be an iterative process. Segmentation (e.g., generation of individual/segmented patient-specific 3D brain sub-representations, such as a patient-specific 3D brain sub-representations representing the putamen) may gradually deform the mean mesh (e.g., gradually deform shape-constrained deformable brain model 100) to match the patient-specific scan (e.g., scan 210). In other words, shape may be constrained to the mean mesh (e.g., constrained to shape-constrained deformable brain model 100), which can grow or shrink without morphing into a different shape.

Where patient-specific 3D brain representation 220 is a voxel-based representation, examples can segment (e.g., extract) individual 3D brain sub-representations by applying a marching cubes algorithm to the 3D brain representation 220. In an illustrative example, a 3D putamen sub-representation may be a 3D boundary surface of the 3D representation for the putamen. In certain cases, examples can generate the 3D putamen sub-representation by applying the marching cubes algorithm to the 3D representation for the brain structure.

Through the above-described adaptation, mesh vertex-based correspondences can be preserved between mesh vertices of shape-constrained deformable brain model 100 and mesh vertices of the patient-specific 3D brain structure mesh representations 220.

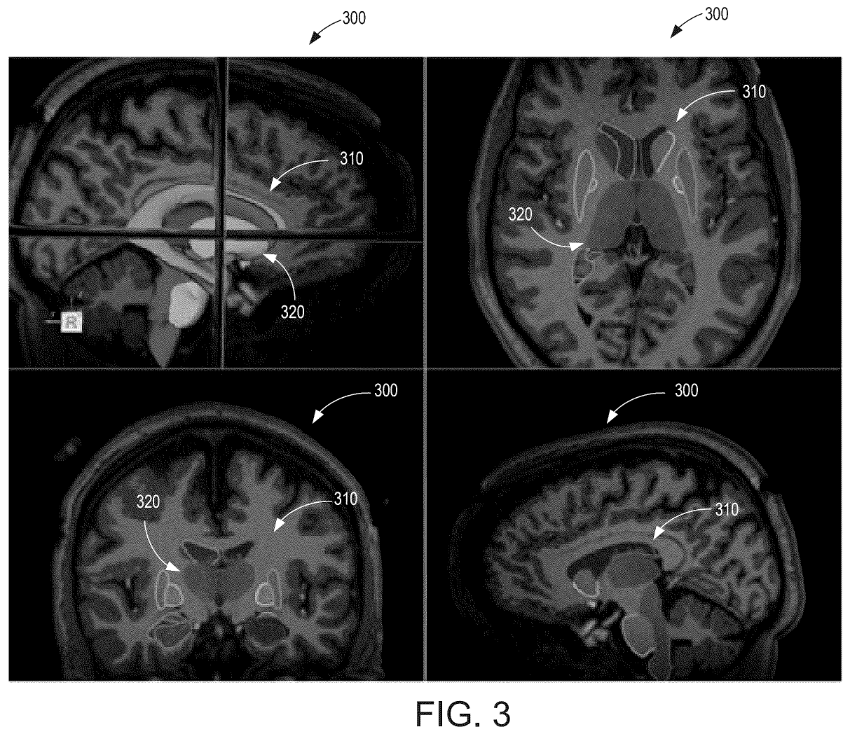

FIG. 3 depicts an example patient-specific segmented 3D brain representation 300, in accordance with various examples of the presently disclosed technology. As described above, patient-specific segmented 3D brain representation 300 may be a computerized 3D representation of a patient's brain comprised of individual 3D structure sub-representations representing various regions/structures of the patient's brain (e.g., sub-cortical structures such as the putamen, thalamus, etc.). Patient-specific segmented 3D brain representation 300 can be based on imaging data (e.g., MRI or CT scans) of the patient's brain, as described above in connection with FIG. 2.

Patient-specific 3D brain representation 300 may be segmented into various 3D sub-representations that represent separate structures/regions of the patient's brain. In the specific example of FIG. 3, patient-specific segmented 3D brain representation 300 includes 3D target brain sub-representation 310 of a region of the patient's brain (i.e., a sub-representation of patient-specific segmented 3D brain representation 300). Here, the target brain region (represented by 3D target brain sub-representation 310) may be a region of the patient's brain targeted for drug/infusate delivery. In some examples, a target brain region may be a specific brain structure (e.g., a sub-cortical structure such as the putamen, thalamus, etc.), a portion/segment of a specific brain structure, or a more general region/volume of the patient's brain. For example, the target brain region may be the 3D target brain sub-representation 310, while in another example the target brain region may be 3D target brain sub-representation 320 (e.g., a putamen). A 3D target brain sub-representation may be used herein to refer to a 3D target brain sub-representation of a target region of the brain (e.g., 3D target brain sub-representation 310) and/or sub-representation of a target structure of the brain (e.g., 3D target brain sub-representation 320)

As described above in connection with FIGS. 1 and 2, examples can generate a patient-specific segmented 3D brain representation (e.g., segmenting patient-specific 3D brain representation 100 into individual structure/region representations) by adapting the generalized segmented 3D brain representation (i.e., shape-constrained deformable brain model 100) to patient-specific brain image data (e.g., one or more scans 210 of a patient's brain). In this way, examples of the presently disclosed technology can be reproduced across different patients, procedures, sites, etc. Accordingly, examples of the presently disclosed technology may improve upon existing infusion coverage visualization and estimation methodologies which are not as easily reproducible.

In certain examples, the generalized segmented 3D brain representation described above may comprise a 3D mesh representation, as described above in connection with FIGS. 1 and 2. A mesh (or surface mesh) may refer to a representation of a larger domain (e.g., a volume or surface) comprised of smaller discrete cells called mesh elements, and mesh vertices at the junctions of adjacent/adjoining mesh elements. Meshes can be used to compute solutions to equations across individual mesh elements, which then can be used to approximate solutions over the larger domain. For example, meshes can be used to compute volumes contained within 3D closed mesh boundary surfaces.

By adapting a generalized segmented 3D (mesh) brain representation to imaging data of the patients' brain, examples can generate patient-specific segmented 3D (mesh) brain representations. These patient-specific segmented 3D (mesh) brain representations may preserve point-based correspondences between mesh vertices of the generalized segmented 3D (mesh) brain representation and mesh vertices of the patient-specific segmented 3D (mesh) brain representations. Such preservation can be used to establish point-based correspondences for target brain regions (e.g., the target brain region represented, for example, by 3D target brain sub-representation 310, 3D target brain sub-representation 320, etc.) for therapy delivery across varied populations.

Referring again to FIG. 3, patient-specific segmented 3D brain representation 300 may comprise a 3D mesh representation. In these examples, the various 3D sub-representations of patient-specific segmented 3D brain representation 300 (e.g., 3D target brain sub-representation 310, 3D target brain sub-representation 320, etc.) may be represented as 3D closed mesh boundary surfaces comprised of mesh elements and mesh vertices (the volumes of these 3D closed mesh boundary surfaces may comprise mesh elements and mesh vertices as well). As will be described below, examples can estimate a volume for a target brain region by computing a volume for a 3D closed mesh boundary surface (e.g., 3D target brain sub-representation 310, 3D target brain sub-representation 320, etc.) representing the target brain region. This estimated volume for the target brain region can be compared to an estimated volume for infusate delivered to the target brain region to estimate a level of coverage for delivered infusate within the target brain region.

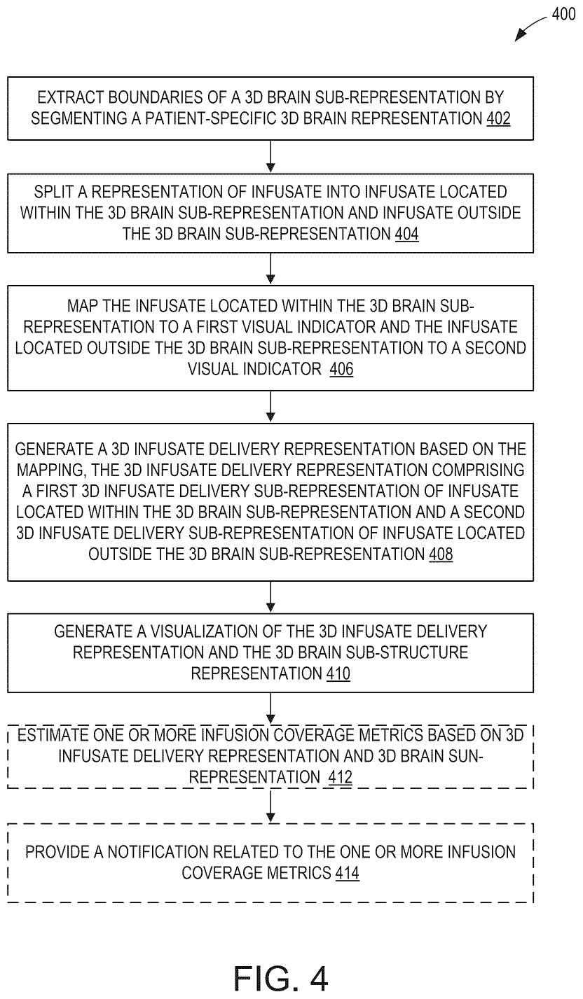

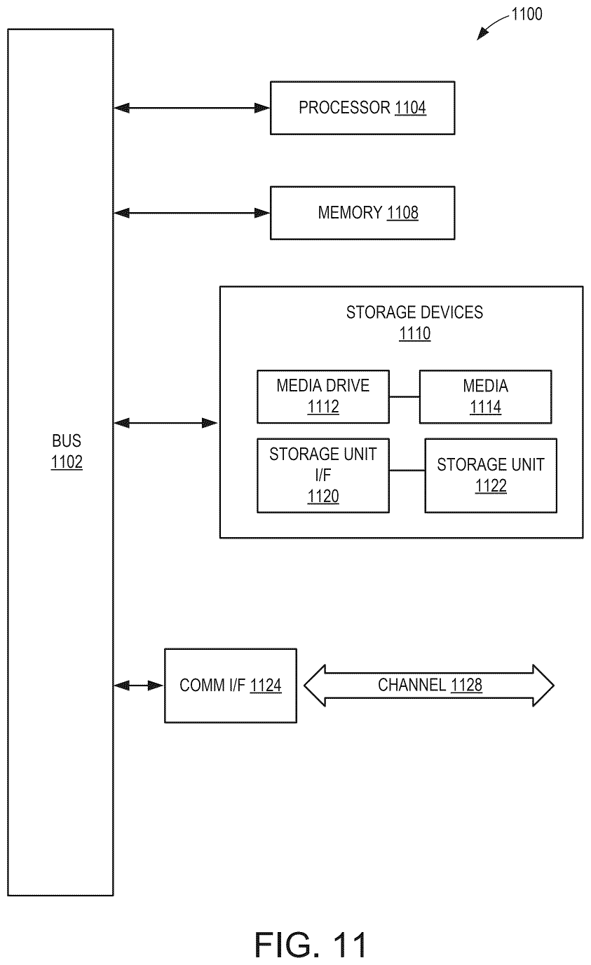

FIG. 4 depicts an example flow diagram of a process 400 that may be used to visualize infusate delivered to a target brain region, in accordance with various examples of the presently disclosed technology. In some examples, process 400 may be used to generate a visualization of one or more infusate coverage metrics of the infusate delivered to a target brain region. Process 400 may be implemented as instructions stored in a memory, which can be executed by a processor to perform the process 400. Process 400 may be executed by a single computing device (e.g., computing component 1100 of FIG. 11) or distributed across multiple computing devices.

At operation 402, boundaries of a 3D brain sub-representation can be extracted by segmenting a patient-specific 3D brain representation. For example, as described in conjunction with FIGS. 1-3, examples can generate a patient-specific 3D brain representation of the patient's brain by adapting a generalized 3D brain representation to patient-specific brain image data. The patient-specific 3D brain representation can be segmented into various 3D brain sub-representations that represent separate structures and/or regions of the patient's brain.

In certain examples, the generalized segmented 3D brain representation described above may comprise a mesh/surface mesh representation, as described above in connection with FIGS. 1-3. In examples, the various 3D brain sub-representations of patient-specific segmented 3D brain representation may be represented as 3D closed mesh boundary surfaces comprised of mesh elements and mesh vertices.

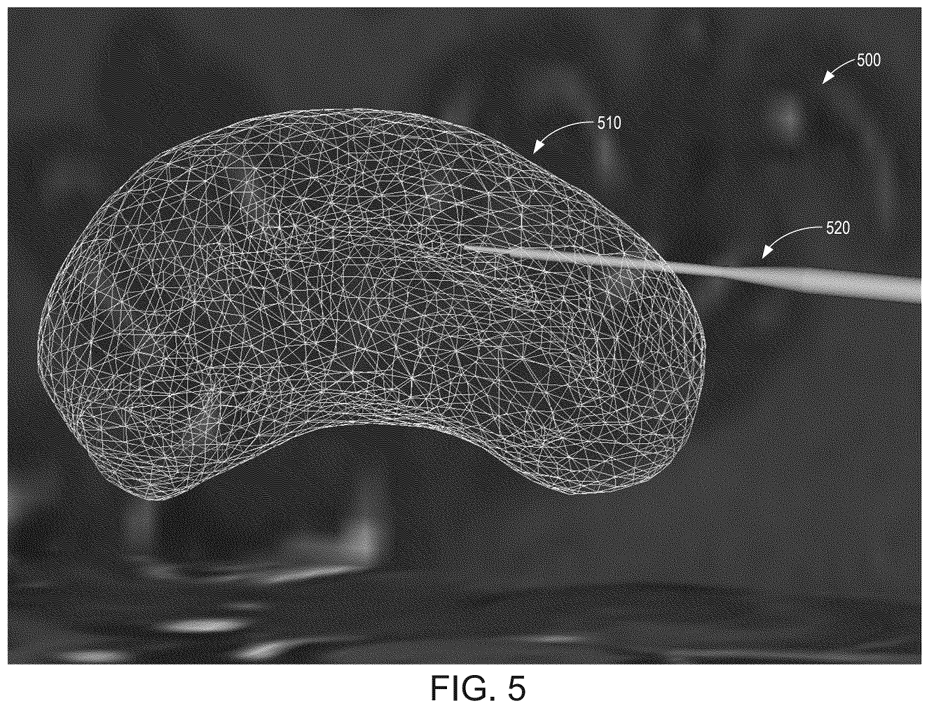

FIG. 5 depicts an example patient-specific segmented 3D brain representation 500, in accordance with an example of the presently disclosed technology. FIG. 5 depicts a zoomed in view of the patient-specific segmented 3D brain representation 500 that focuses on a target brain sub-structure/region. In this example, the target brain sub-structure/region is a putamen. However, embodiments disclosed herein may be applicable to any target sub-structure/region of a patient's brain.

Like patient-specific segmented 3D brain representation 300 of FIG. 3, patient-specific segmented 3D brain representation 500 comprises a 3D brain sub-representation 510 for the target brain region (sometimes referred to herein as a 3D target brain sub-representation). Here patient-specific segmented 3D brain representation 500 and 3D target brain region sub-representation 510 may be the same/similar as patient-specific segmented 3D brain representation 300 and 3D target brain sub-representation 310, respectively. As an example, patient-specific segmented 3D brain sub-representation 510 is a 3D representation of the target brain sub-structure/region (e.g., the putamen).

As depicted, patient-specific segmented 3D brain representation 500 and 3D brain sub-representation 510 may comprise 3D mesh (surface mesh) representations. The 3D mesh representations may be represented as 3D closed mesh boundary surfaces comprised of mesh elements and mesh vertices. The mesh elements and mesh vertices, which form voxels that define the 3D closed mesh boundary surfaces, such as the 3D closed mesh boundary surface of the 3D brain sub-representation 510. The voxels, including the mesh elements and mesh vertices, of the 3D closed mesh boundary corresponding to the target brain sub-structure/region (e.g., voxels of the 3D brain sub-representation 510) may be extracted from the patient-specific segmented 3D brain representation 500 and stored in a data store or other memory for use in process 400.

As depicted in FIG. 5, a targeting cannula 550 may be used to deliver infusate to the target brain region represented by 3D brain sub-representation 510, in some examples.

Returning to FIG. 4, at operation 404, a infusate delivery representation of infusate delivered to the target brain sub-structure/region (sometimes referred to herein as a representation of infusate) can be split into sub-representations. For example, a infusate delivery representation of infusate delivered to the target brain sub-structure/region can be generated and split into infusate into infusate located within the 3D brain sub-representation (sometimes referred to herein as first infusate delivery sub-representation) and infusate outside of the 3D brain sub-representation (sometimes referred to herein as second infusate delivery sub-representation). For example, a infusate delivery representation can be split into infusate located outside and infusate located inside based on the boundaries of the 3D brain sub-representation. In some cases, there may be one or more regions of infusate location inside and/or outside of target brain sub-structure/region. As such, the infusate delivery representation can be split into one or more first sub-representations and one or more second sub-representation, where each sub-representations corresponds to a region of delivered infusate that is distinct from other regions of delivered infusate.

In an illustrative example, operation 404 can include detecting intersections of the representation of infusate with the boundaries of the 3D brain sub-representation. The intersections can distinguish between the infusate delivered to the target brain sub-structure/region inside the 3D brain sub-representation and the infusate delivered to the target brain sub-structure/region located outside the 3D brain sub-representation. As described above, the 3D brain sub-representation may be represented as 3D closed mesh boundary surfaces comprised of mesh elements and mesh vertices. By applying an intersection algorithm, as known in the art, to the representation of the infusate delivered to the target brain sub-structure/region, intersections between the representation of delivered infusate with the 3D closed mesh boundary surfaces can be detected. From the detected intersections, the infusate delivery representation can be split into infusate inside the 3D brain sub-representation and infusate outside the 3D brain sub-representation.

Examples herein can obtain the infusate delivery representation through a variety of approaches. These approaches can provide representations of where the infusate propagates upon delivery to the target brain sub-structure/region. The representations, at this stage, may not distinguish between infusate located inside from infusate located outside of the of the target brain sub-structure/region.

In one approach example, a infusate delivery representation can be obtained by comparing a first image/scan (e.g., MRI scan or CT scan obtained during an infusion procedure) of a patient's brain acquired after infusate has been delivered to the patient's brain to a second (e.g., baseline) image/scan of the patient's brain acquired at a point in time prior to the first image/scan. The second image/scan may be acquired prior to any infusate has been delivered or acquired after a portion of infusate has been delivered, but before the first image/scan is acquired. In various examples, the comparison may comprise subtracting the first image/scan from the second/scan image. In some cases, the images/scans may be 3D images of the patient's brain during the infusion. In this case, subtraction may refer to a process of subtracting numeric values of voxels of one 3D image from numeric values of voxels of another 3D image-such subtraction can be used to identify differences between the two 3D images. Based on this comparison/subtraction, examples can generate a representation of infusate delivered to the target brain region/structure that volumetrically/spatially represents the delivered infusate within the patient's brain. Additional details on this approach can be found, for example, in U.S. application Ser. No. 17/894,589, the disclosure of which is incorporated herein by reference in its entirety.

In another example, the infusate delivered to the target brain sub-structure/region can be simulated, for example, by a biophysical simulation of infusion. In this case, embodiments disclosed herein may obtain simulation data, which can include infusate data that represents the infusate prior to infusion and propagation characteristics of the infusate (e.g., viscosity data, capillary action data, surface tension, total volume of infusate, etc.). The infusate data may be used to simulate the propagation of the delivered infusate within a volume. The simulation data may also include the 3D brain 3D brain representation and 3D brain sub-representations. The simulation data can be applied to a biophysical simulation, such as a finite element model, which simulates an infusion by inserting a simulated cannula (e.g., cannula 550) inserted at a desired point in the 3D brain structure. The simulation then propagates the infusate through simulation to generate the representation of the infusate delivered to the target brain sub-structure/region.

Regardless of the approach used to obtain the infusate delivery representation of infusate delivered to the target brain sub-structure/region, the representation may be provided as a 3D infusate delivery representation. The 3D infusate delivery representation may be a computerized 3D representation that volumetrically and spatially represents the delivered infusate within the patient's brain. The 3D infusate delivery representation can be provided as a voxel-based representation of the delivered infusate, which can be transformed into a 3D mesh (surface mesh) representation of the delivered infusate. Accordingly, similar to the 3D brain representation and 3D brain sub-representations described above, the 3D infusate delivery representation may be represented as 3D closed mesh boundary surfaces comprised of mesh elements and mesh vertices (the volumes of these 3D closed mesh boundary surfaces may comprise mesh elements and mesh vertices as well).

The delivered infusate within the patient's brain will typically include a volume of delivered infusate located in the target brain region, but in some cases, may also include a volume of delivered infusate located outside the target brain region. At this stage, the computerized estimation may not be able to determine, from the 3D infusate delivery representation alone, which volumes/portions of the 3D infusate delivery representation correspond to delivered infusate within the target brain region vs. which volumes/portions of the 3D infusate delivery representation correspond to delivered infusate located outside of the target brain region. Accordingly, as alluded to above, operation 404 can include detecting intersections of the 3D infusate delivery representation with the boundaries of the 3D brain sub-representation. The 3D infusate delivery representation can be segmented (e.g., split) into the first infusate delivery sub-representation and the second infusate delivery sub-representation. For example, an intersection algorithm can be applied to the 3D closed mesh boundary surfaces of the 3D infusate delivery representation and the 3D brain sub-representation to detected intersections therebetween. The 3D infusate delivery representation can then be divided into sub-representations of infusate located outside the 3D brain sub-representation and infusate located outside the 3D brain sub-representation.

The segmented/split representation of the infusate delivered to the target brain sub-structure/region can be provided as a bitmask volume. In an example, the infusate delivery representation can be assigned numerical values according to the segmentation. For example, a first numerical value can be assigned to the first infusate delivery sub-representation of infusate located within the 3D brain sub-representation and a second numerical value can be assigned to the second infusate delivery representation of infusate located outside the 3D brain sub-representation. In the case of voxel-based representations, each voxel associated a first 3D infusate delivery sub-representation of infusate located within the 3D brain sub-representation can be assigned the first numerical value (e.g., “1”) and each voxel associated a second 3D infusate delivery sub-representation of infusate located outside the 3D brain sub-representation can be assigned the second numerical value (e.g., “0”). 0 and 1 are used as examples, any numerical value may be used to generate the bitmask volume.

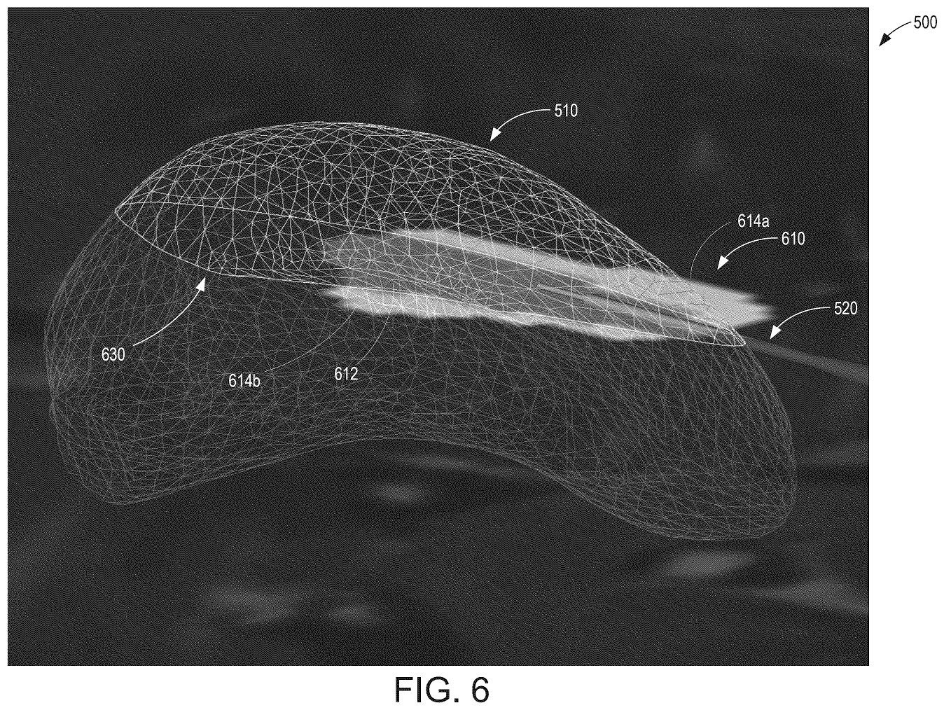

FIG. 6 depicts an example visualization of a 2D slice of a segmented/split infusate delivery representation 610 delivered to the target brain sub-structure/region according to examples of the presently disclosed technology combined with patient-specific segmented 3D brain representation 500. FIG. 6 depicts the zoomed in view of the patient-specific segmented 3D brain representation 500 of FIG. 5 combined/overlaid with a visualization of the segmented/split infusate delivery representation 610. The segmented/split infusate delivery representation 610 is illustratively shown as a 2D slice of a 3D infusate delivery representation taken along a plane 630 (shown as a perimeter of 3D brain sub-representation 510).

In this example, the infusate delivery representation 610 is split into a plurality of infusate delivery sub-representations, as described above. As shown in FIG. 6, segmenting the infusate delivery representation 610 provides a first infusate delivery sub-representation 612 of infusate located inside the 3D brain sub-representation 510 and second infusate delivery sub-representations 614a and 614b of infusate outside the 3D brain sub-representation 510. FIG. 6 depicts a visualization of a bitmask volume for segmented/split infusate delivery sub-representation 610, where first infusate delivery sub-representation 612 is assigned a first numerical value and second infusate delivery sub-representations 614a and 614b are assigned a second numerical value. In another example, second infusate delivery sub-representations 614a and 614b may be assigned different numerical values.

Returning to FIG. 4, at operation 406, the infusate located within the 3D brain sub-representation (e.g., first infusate delivery sub-representation(s)) can be mapped to a first visual indicator and the infusate located outside the 3D brain sub-representation (e.g., second infusate delivery sub-representation(s)) can be mapped to a second visual indicator. In examples, a visual indicator lookup table can be accessed to facilitate mapping infusate located within the 3D brain sub-representation to a first visual indicator and infusate located outside the 3D brain sub-representation to a second visual indicator. The visual indicator lookup table may comprise associations of visual indicators to numerical values. From the visual indicator look up table, a first visual indicator can be assigned to the infusate located within the 3D brain sub-representation based on the first numerical value and the second visual indictor can be assigned to the infusate outside the 3D brain sub-representation based on the second numerical value. That is, the first and/or second numerical values can be used to retrieve a first and/or second visual indicator, respectively, from the visual indicator lookup table according to the associations therein. In the case of multiple infusate delivery sub-representations, for example where there are a plurality of second infusate delivery sub-representations, each sub-representation may be assigned different visual indicators if desired.

Visual indicators may include any visual attribute that can be used to visually distinguish between neighboring regions of in visual medium (e.g., display). In various examples, visual indicators may be provided a different colors associated with different numerical values (e.g., the first visual indicator may be green and the second visual indicator may be red). In another example, different line types of fills may be used as visual indicators. For example, one visual indicator may be a solid volume, while another visual indicator may be hatch fill, or the like. In yet another example, different strobing or pulsing patterns may be used. Any distinguishing visual indicators can be used.

FIG. 6 also illustrates an example visualization of first and second infusate delivery sub-representations mapped to first and second visual indicators, respectively. In this case, the first infusate delivery sub-representation 612, representing infusate located within the 3D brain sub-representation 510, is assigned a first visual indicator (e.g., green in some examples, but illustratively shown as a dark gray). The second infusate delivery sub-representations 614a and 614b, representing infusate located outside the 3D brain sub-representation 510, are assigned a second visual indicator (e.g., red in some examples, but illustratively shown as a light gray).

Referring back to FIG. 4, at operation 408, a 3D infusate delivery representation is generated based on the mapping from operation 406 and the segmented/split infusate delivery representations from operation 404. In examples, generating the 3D infusate delivery representation may include extracting boundaries of the 3D surfaces meshes (e.g., voxels) of the 3D infusate delivery sub-representations and visual indicator mapped thereto. The 3D infusate delivery representation can comprise a first 3D infusate delivery sub-representation of infusate located within the 3D brain sub-representation and a second 3D infusate delivery sub-representation of infusate located outside the 3D brain sub-representation. In examples, first 3D infusate delivery sub-representation and second 3D infusate delivery sub-representation can be generated (e.g., extracted) from the first and second sub-representations of infusate, as described above. For example, the first 3D infusate delivery sub-representation of infusate located within the 3D brain sub-representation can be extracted from the first infusate delivery sub-representation of the bitmask volume, and the second 3D infusate delivery sub-representation can be extracted from the second sub-representation of infusate located outside the 3D brain sub-representation as defined by the bitmask volume.

In examples, the first and second infusate delivery sub-representations (e.g., as defined in the bitmask) can be meshed to provide 3D closed mesh boundary surfaces. For example, the extracted voxels of the first infusate delivery sub-representation can be meshed to provide a first 3D closed mesh boundary surface representative of infusate located within the 3D brain sub-representation (e.g., first 3D infusate delivery sub-representation). Examples can apply a marching cubes algorithm to the bitmask to generate the first 3D closed mesh boundary. The second infusate delivery sub-representation can be similarly meshed to provide second 3D closed mesh boundary surfaces representative of infusate located outside of the 3D brain sub-representation (e.g., second 3D infusate delivery sub-representations). As described above, the 3D closed mesh boundary surfaces comprise of mesh elements and mesh vertices that can be used to compute a volume for the 3D closed mesh boundary surface.

At operation 410, a visualization of the 3D infusate delivery representation and the 3D brain sub-representation can be generated. For example, the 3D infusate delivery representation obtained at operation 408 can be combined/overlaid with the 3D brain sub-representation. The first 3D meshed boundary surface for the infusate located within the 3D brain sub-representation and second 3D meshed boundary surface for infusate located outside of the 3D brain sub-representation can overlaid with the 3D meshed boundary surface of the 3D brain sub-representation. A visualization of the combined/overlaid 3D mesh boundary surfaces can then be generated (e.g., rendered from voxels) and presented via a display. The visual indicators mapped at operation 406 can be maintained in the visualization, such that the first 3D meshed boundary surface can be displayed using the first visual indicator and the second 3D meshed boundary surface can be displayed using a second visual indicator. The 3D mesh boundary surface of the 3D brain sub-representation can be assigned and displayed using a third visual indicator to distinguish from the 3D infusate delivery sub-representations.

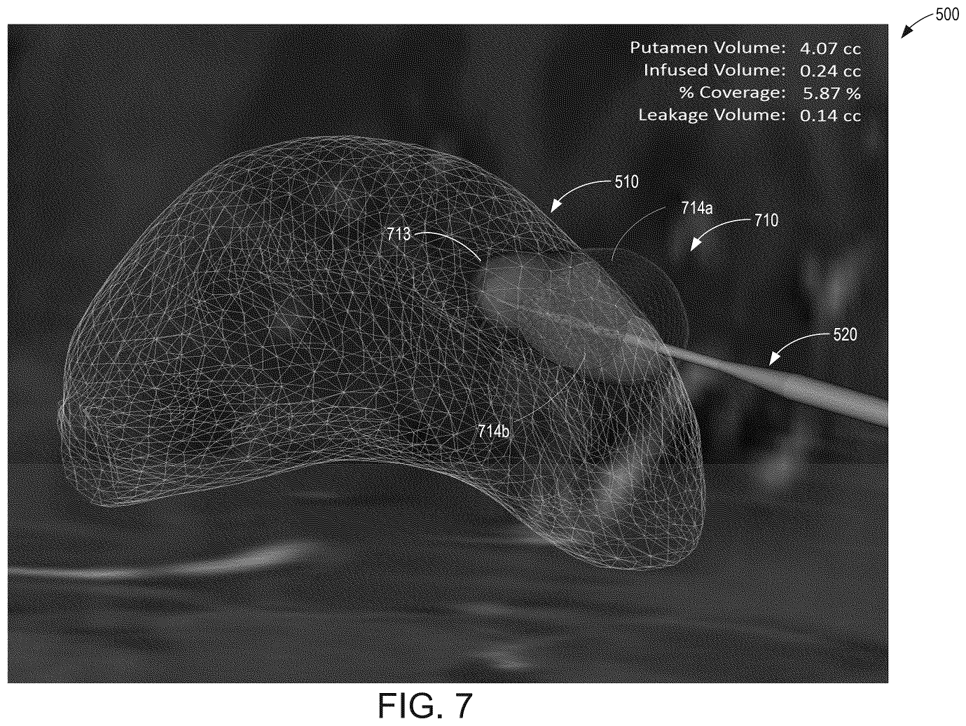

FIG. 7 depicts an example visualization of a 3D infusate delivery representation 710 combined with the 3D brain structure representation 500 of FIG. 5 generated in accordance with the presently disclosed technology. FIG. 7 depicts the zoomed in view of the patient-specific segmented 3D brain representation 500 that focuses on a target brain sub-structure/region. In this example, the target brain sub-structure/region s a putamen. However, embodiments disclosed herein may be applicable to any target sub-structure/region of a patient's brain represented as 3D brain sub-structure 510.

As depicted, 3D brain sub-representation 510 and the 3D infusate delivery representation 710 may comprise 3D mesh (surface mesh) representations combined/overlaid with each other. As described above, the 3D mesh representations may be represented as 3D closed mesh boundary surfaces comprised of mesh elements and mesh vertices. The mesh elements and mesh vertices, which form voxels that define the 3D closed mesh boundary surfaces. Voxels, including the mesh elements and mesh vertices, of the 3D closed mesh boundary corresponding to the target brain sub-structure/region (e.g., voxels of the 3D brain sub-representation 510) may be extracted from the patient-specific segmented 3D brain representation 500. Similarly, voxels, including the mesh elements and mesh vertices, of the 3D closed mesh boundary corresponding to the infusate located within the 3D brain sub-representation 510 (e.g., voxels of the first 3D meshed boundary surface 712) and the 3D closed mesh boundary corresponding to the infusate located outside the 3D brain sub-representation 510 (e.g., voxels of the second 3D meshed boundary surfaces 714a and 714b) may be extracted from the 3D infusate delivery representation 710. The various voxels can be stored in a data store or other memory for use downstream.

In examples, the 3D mesh (surface mesh) representations of the 3D brain sub-representation 510 and the 3D infusate delivery representation 710 can be visually presented on a display. For example, a graphical user interface (GUI) (e.g., executed by computing component 1100 of FIG. 11) may be provided that generates one or more display viewports that include graphical visual representations of the 3D brain sub-representation 510 and the 3D infusate delivery representation 710. In an example, FIG. 7 may be illustrative of such a viewport that can be used to present a graphical visual representation of the 3D brain sub-representation 510 and the 3D infusate delivery representation 710. In this case, the 3D mesh (surface mesh) representations are graphically displayed by the GUI. The GUI may be used for visually reviewing and monitoring of infusion. In this case, the mesh elements of the various 3D mesh representation comprise triangles, but in other examples mesh elements may comprise different shape.

Accordingly, examples can utilize these 3D infusate delivery representations to track/visualize infusion coverage during an infusion procedure, either in real-time or in simulating a planned infusions. For example, by comparing the 3D infusate delivery representation to a patient-specific segmented 3D brain representation through a visual medium, examples can a visual representation of infusion coverage metrics that a neurosurgeons can use to estimate infusion performance. Example infusion coverage metrics include, but are not limited to, infused volume (e.g., volume of infusate located within the target brain sub-structure/region), coverage percentage or level of infusion coverage (e.g., percentage volume of target brain sub-structure/region infused with infusate), and leakage volume (e.g., volume of infusate located outside of the target brain sub-structure/region), among others.

Returning to FIG. 4, at optional operation 412, examples can estimate one or more infusion coverage metrics for delivered infusate within the target brain region based on 3D infusate delivery representation and 3D brain sub-structure. Examples can compare the 3D infusate delivery representation from operation 408 with 3D brain sub-structure from operation 402 to estimate one or more of: infused volume based on the 3D infusate delivery sub-representation of the infusate located within the 3D brain sub-structure; coverage percentage by comparing the estimated infused volume to a volume for the target brain region estimate based on the 3D brain sub-structure; and leakage volume based on the 3D infusate delivery sub-representation of the infusate located outside of the 3D brain sub-structure.

Examples can use various techniques to estimate a volume of a 3D representation (e.g., first 3D infusate delivery sub-representation for infusate located within the target brain sub-structure, second 3D infusate delivery sub-representation of infusate located outside of the target brain sub-structure, and 3D brain sub-representation for the target brain sub-structure). For instance, examples can count a number of voxels of a respective 3D representation and translate the number of voxels to a unit of volume. A conversion factor for a number of voxels to a unit of volume (e.g., cubic centimeter or “cc” in some examples) can be derived from the patient-specific 3D brain structure and image data used to adapt the generalized 3D brain structure to the patient-specific 3D brain structure. Volumes may also be estimated using 3D surfaces (e.g., 3D closed mesh boundary surfaces) instead of voxel counting. This could involve estimating a 3D surface for a 3D infusate delivery sub-representation (e.g., a 3D closed mesh boundary surface for the 3D infusate delivery representation), and comparing this 3D surface to a 3D surface (e.g., a 3D closed mesh boundary surface) for the 3D target brain region sub-representation. In other instances, volumes for a 3D representation can be estimated by computing a volume for a respective 3D closed mesh boundary surface. For example, a total mesh volume could be computed using signed tetrahedral volumes. One volume may be computed for each mesh triangle, using a common origin to form each tetrahedron. Using any of the above techniques, examples can compute: a volume of infusate inside the target brain sub-structure/region can be estimated from the first 3D infusate delivery sub-representation of infused located inside the 3D brain sub-representation; a volume of infusate outside of the target brain sub-structure/region (also known as leakage volume) can be estimated from the second 3D infusate delivery sub-representation of infused located outside the 3D brain sub-representation; and a volume of the target brain sub-structure/region can be estimated from the 3D brain sub-representation.

Examples can estimate coverage percentage based on the volume of estimated from the first 3D infusate delivery sub-representation of infused located inside the 3D brain sub-representation. For example, the estimated volume for infusate located within the target brain region can be compared to the estimate volume of the target brain region. For instance, examples can divide (e.g., a ratio of) the estimated volume for delivered infusate within the target brain region by the estimated volume for the target brain region (and multiple by 100%) to obtain a percentage of infusate coverage within the target brain region.

In some examples, one or more infusion coverage metrics estimated at operation 412 can be combined/overlaid with the visualization generated at operation 410. For example, as shown in FIG. 7, legend can be generated that displays the infusion coverage metrics. In this example, legend includes an estimated volume of the target brain sub-structure/region (e.g., Putamen Volume: 4.07 cc); an estimate infused volume (e.g., Infused Volume: 0.24 cc); an estimate coverage percentage (e.g., % Coverage 5.87%); and an estimate leakage volume (e.g., Leakage Volume: 0.14 cc).

Process 400 can also include optional operation 414, which can provide a notification related to the estimated one or more infusion coverage metrics. In various instances, this operation may comprise providing a notification related to one or more of: the infused volume; coverage percentage; and leakage volume.

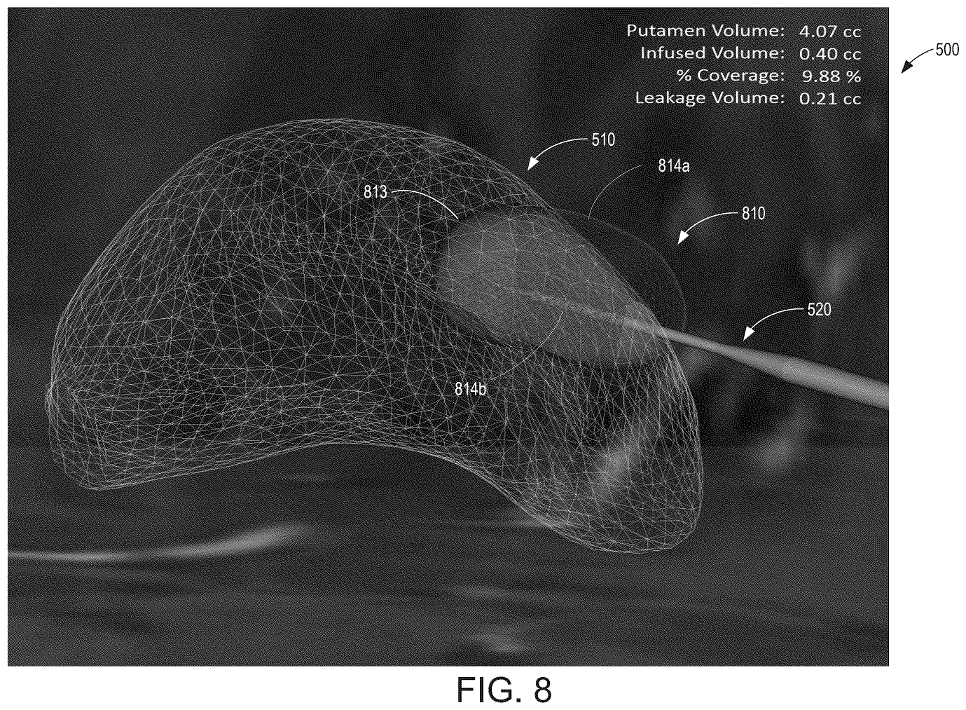

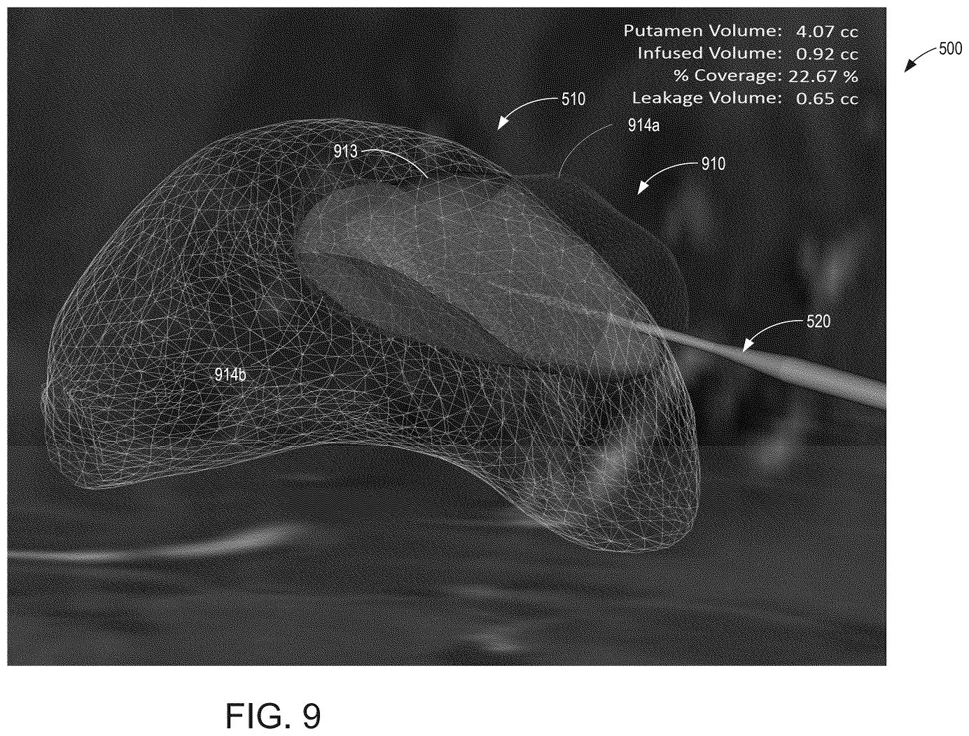

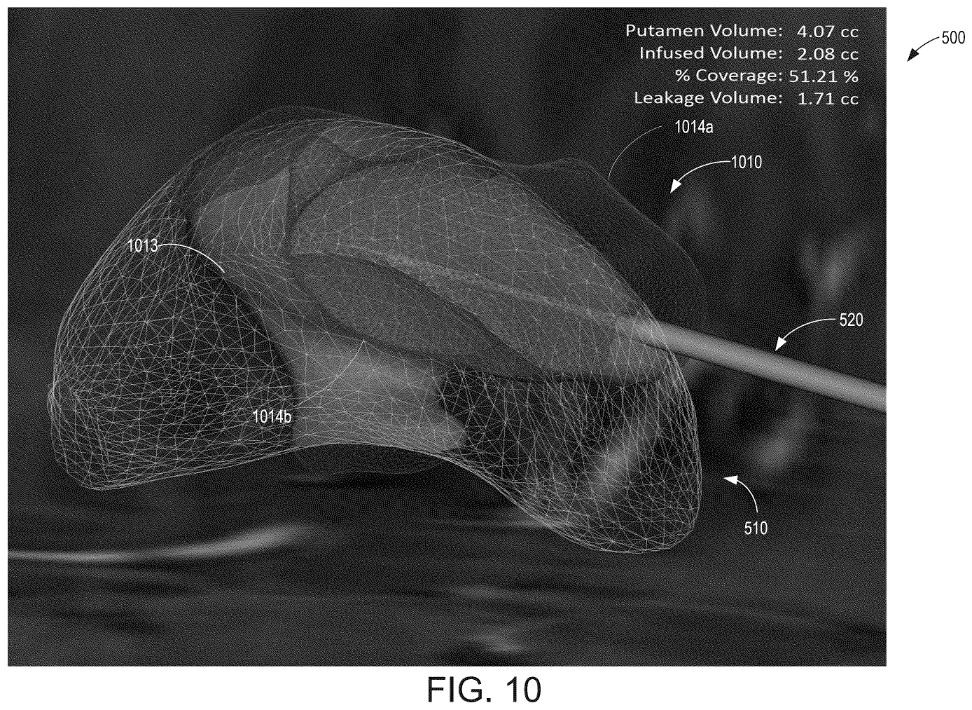

The provided notification may take various forms that highlight an infusion coverage metric for a user/neurosurgeon. In one instance, the notification can highlight a infusion coverage metric (e.g., estimated coverage percentage) using a “traffic light” notion: e.g., red (e.g., complete coverage achieved/stop infusion), yellow (e.g., nearly complete coverage achieved/continue infusion with caution), and green (e.g., incomplete infusion coverage/continue infusion). Here, infusion can be stopped when a certain percentage of coverage for the targeted brain region has been achieved (as indicated by the “red symbol”). As described above, the provided notification may also comprise an notification/alert when delivered infusate is leaking/located outside of the target brain region. Such an alert may comprise, e.g., a recommendation to stop infusion or to adjust the location of the targeting cannula delivering infusate. The alert/notifications can be used in conjunction with the 3D representations generated at operation 410. FIGS. 8-11 depict example visualizations of various time points during an infusion-either real-time or simulated-generated in accordance with the presently disclosed technology. FIGS. 8-11 illustrate time points occurring, for example, after the time point of the infusion shown in FIG. 7. In the example, FIG. 7 depicts a visualization generated for a first time point comprising 3D infusate delivery representation 710 combined/overlaid with the 3D brain structure representation 500. FIG. 8 depicts a visualization generated for a second time point, after the first time point, comprising 3D infusate delivery representation 810 (e.g., first 3D meshed boundary surface 812 of infusate located within the 3D brain sub-structure 510 and second 3D meshed boundary surfaces 814a and 814b of infusate located outside of the 3D brain sub-structure 510) combined/overlaid with the 3D brain structure representation 500. FIG. 9 depicts a visualization generated for a third time point, after the second time point, comprising 3D infusate delivery representation 910 (e.g., first 3D meshed boundary surface 912 of infusate located within the 3D brain sub-structure 510 and second 3D meshed boundary surfaces 914a and 914b of infusate located outside of the 3D brain sub-structure 510) combined/overlaid with the 3D brain structure representation 500. FIG. 10 depicts a visualization generated by a GUI at a second time point, after the first time point, comprising 3D infusate delivery representation 1010 (e.g., first 3D meshed boundary surface 1012 of infusate located within the 3D brain sub-structure 510 and second 3D meshed boundary surfaces 1014a and 1014b of infusate located outside of the 3D brain sub-structure 510) combined/overlaid with the 3D brain structure representation 500. Thus, FIGS. 7-10 depict various stages of an infusion procedure along a time horizon, where the amount of infusate delivered to the target brain sub-structure increases accordingly. As a result, in the examples of FIG. 7-10, the infusion coverage metrics also change, as shown in each figure.

While the examples depicted in FIGS. 5-11 are described with reference to a single target brain structure and thus two infusate delivery sub-representations, embodiments of the presently disclosed technology are not so limited. Examples may provide any number of infusate delivery sub-representations, each associated with a different visual indicator. As an illustrative example, an infusate delivery representation may be split at operation 404 of FIG. 4 into one or more first infusate delivery sub-representations of infusate located inside the target brain sub-structure/region; one or more second infusate delivery sub-representations of infusate located outside the target brain sub-structure/region; and one or more third infusate delivery sub-representations of infusate that propagated into a brain sub-structure/region neighboring the target brain sub-structure/region. The infusate delivery representation may include sub-representations of infusate that propagated into any brain sub-structures/region other than the target brain sub-structure/region, where each representation may have the same or different visual indicator. In this case, each first, second, and third infusate delivery sub-representation may be mapped to different visual indicators and used to generated first, second, and third 3D infusate delivery sub-representations (e.g., operations 406-408 of FIG. 4), which can then be used to generated a visualization of the 3D infusate delivery representation (e.g., operation 410) and estimate coverage metrics (e.g., operation 412).

As used herein, the terms circuit and component might describe a given unit of functionality that can be performed in accordance with one or more examples of the present application. As used herein, a component might be implemented utilizing any form of hardware, software, or a combination thereof. For example, one or more processors, controllers, ASICs, PLAS, PALs, CPLDs, FPGAs, logical components, software routines or other mechanisms might be implemented to make up a component. Various components described herein may be implemented as discrete components or described functions and features can be shared in part or in total among one or more components. In other words, as would be apparent to one of ordinary skill in the art after reading this description, the various features and functionality described herein may be implemented in any given application. They can be implemented in one or more separate or shared components in various combinations and permutations. Although various features or functional elements may be individually described or claimed as separate components, it should be understood that these features/functionality can be shared among one or more common software and hardware elements. Such a description shall not require or imply that separate hardware or software components are used to implement such features or functionality.

Where components are implemented in whole or in part using software, these software elements can be implemented to operate with a computing or processing component capable of carrying out the functionality described with respect thereto. One such example computing component is shown in FIG. 11. Various examples are described in terms of this example-computing component 1100. After reading this description, it will become apparent to a person skilled in the relevant art how to implement the application using other computing components or architectures.

Referring now to FIG. 11, computing component 1100 (also referred to as a computing system) may represent, for example, computing or processing capabilities found within a self-adjusting display, desktop, laptop, notebook, and tablet computers. They may be found in hand-held computing devices (tablets, PDA's, smart phones, cell phones, palmtops, etc.). They may be found in workstations or other devices with displays, servers, or any other type of special-purpose or general-purpose computing devices as may be desirable or appropriate for a given application or environment. Computing component 1100 might also represent computing capabilities embedded within or otherwise available to a given device. For example, a computing component might be found in other electronic devices such as, for example, portable computing devices, and other electronic devices that might include some form of processing capability.

Computing component 1100 might include, for example, one or more processors, controllers, control components, or other processing devices. Processor 1104 might be implemented using a general-purpose or special-purpose processing engine such as, for example, a microprocessor, controller, or other control logic. The processor 1104 may be implemented in hardware, software, or combinations thereof. Processor 1104 may be connected to a bus 1102. However, any communication medium can be used to facilitate interaction with other components of computing component 1100 or to communicate externally.

Computing component 1100 might also include one or more memory components, simply referred to herein as main memory 1108. For example, random access memory (RAM) or other dynamic memory, might be used for storing information and instructions to be executed by processor 1104. Main memory 1108 may be a non-transitory computer readable medium. Main memory may store instructions, for example, for executing one or more of the operations described in connection with FIG. 4. Main memory 1108 might also be used for storing temporary variables or other intermediate information during execution of instructions to be executed by processor 1104. Computing component 1100 might likewise include a read only memory (“ROM”) or other static storage device coupled to bus 1102 for storing static information and instructions for processor 1104.

The computing component 1100 might also include one or more various forms of information storage mechanism 1110, which might include, for example, a media drive 1112 and a storage unit interface 1120. The media drive 1112 might include a drive or other mechanism to support fixed or removable storage media 1114. For example, a hard disk drive, a solid-state drive, a magnetic tape drive, an optical drive, a compact disc (CD) or digital video disc (DVD) drive (R or RW), or other removable or fixed media drive might be provided. Storage media 1114 might include, for example, a hard disk, an integrated circuit assembly, magnetic tape, cartridge, optical disk, a CD or DVD. Storage media 1114 may be any other fixed or removable medium that is read by, written to or accessed by media drive 1112. As these examples illustrate, the storage media 1114 can include a computer usable storage medium having stored therein computer software or data.

In alternative examples, information storage mechanism 1110 might include other similar instrumentalities for allowing computer programs or other instructions or data to be loaded into computing component 1100. Such instrumentalities might include, for example, a fixed or removable storage unit 1122 and an interface 1120. Examples of such storage units 1122 and interfaces 1120 can include a program cartridge and cartridge interface, a removable memory (for example, a flash memory or other removable memory component) and memory slot. Other examples may include a PCMCIA slot and card, and other fixed or removable storage units 1122 and interfaces 1120 that allow software and data to be transferred from storage unit 1122 to computing component 1100.

Computing component 1100 might also include a communications interface 1124. Communications interface 1124 might be used to allow software and data to be transferred between computing component 1100 and external devices. Examples of communications interface 1124 might include a modem or softmodem, a network interface (such as Ethernet, network interface card, IEEE 802.XX or other interface). Other examples include a communications port (such as for example, a USB port, IR port, RS232 port Bluetooth® interface, or other port), or other communications interface. Software/data transferred via communications interface 1124 may be carried on signals, which can be electronic, electromagnetic (which includes optical) or other signals capable of being exchanged by a given communications interface 1124. These signals might be provided to communications interface 1124 via a channel 1128. Channel 1128 might carry signals and might be implemented using a wired or wireless communication medium. Some examples of a channel might include a phone line, a cellular link, an RF link, an optical link, a network interface, a local or wide area network, and other wired or wireless communications channels.

In this document, the terms “computer program medium” and “computer usable medium” are used to generally refer to transitory or non-transitory media. Such media may be, e.g., memory 1108, storage unit 1120, media 1114, and channel 1128. These and other various forms of computer program media or computer usable media may be involved in carrying one or more sequences of one or more instructions to a processing device for execution. Such instructions embodied on the medium, are generally referred to as “computer program code” or a “computer program product” (which may be grouped in the form of computer programs or other groupings). When executed, such instructions might enable the computing component 1100 to perform features or functions of the present application as discussed herein.

It should be understood that the various features, aspects and functionality described in one or more of the individual examples are not limited in their applicability to the particular example with which they are described. Instead, they can be applied, alone or in various combinations, to one or more other examples, whether or not such examples are described and whether or not such features are presented as being a part of a described example. Thus, the breadth and scope of the present application should not be limited by any of the above-described exemplary examples.

Terms and phrases used in this document, and variations thereof, unless otherwise expressly stated, should be construed as open ended as opposed to limiting. As examples of the foregoing, the term “including” should be read as meaning “including, without limitation” or the like. The term “example” is used to provide exemplary instances of the item in discussion, not an exhaustive or limiting list thereof. The terms “a” or “an” should be read as meaning “at least one,” “one or more” or the like; and adjectives such as “conventional,” “traditional,” “normal,” “standard,” “known.” Terms of similar meaning should not be construed as limiting the item described to a given time period or to an item available as of a given time. Instead, they should be read to encompass conventional, traditional, normal, or standard technologies that may be available or known now or at any time in the future. Where this document refers to technologies that would be apparent or known to one of ordinary skill in the art, such technologies encompass those apparent or known to the skilled artisan now or at any time in the future.

The presence of broadening words and phrases such as “one or more,” “at least,” “but not limited to” or other like phrases in some instances shall not be read to mean that the narrower case is intended or required in instances where such broadening phrases may be absent. The use of the term “component” does not imply that the aspects or functionality described or claimed as part of the component are all configured in a common package. Indeed, any or all of the various aspects of a component, whether control logic or other components, can be combined in a single package or separately maintained and can further be distributed in multiple groupings or packages or across multiple locations.

The terms “optimize,” “optimal” and the like as used herein can be used to mean making or achieving performance as effective or perfect as possible. However, as one of ordinary skill in the art reading this document will recognize, perfection cannot always be achieved. Accordingly, these terms can also encompass making or achieving performance as good or effective as possible or practical under the given circumstances, or making or achieving performance better than that which can be achieved with other settings or parameters.

Additionally, the various examples set forth herein are described in terms of exemplary block diagrams, flow charts and other illustrations. As will become apparent to one of ordinary skill in the art after reading this document, the illustrated examples and their various alternatives can be implemented without confinement to the illustrated examples. For example, block diagrams and their accompanying description should not be construed as mandating a particular architecture or configuration.

1. A method, comprising:
extracting boundaries of a 3D brain sub-representation by segmenting a patient-specific 3D brain representation, the 3D brain sub-representation representing a target structure or region of a brain of a patient;
based on the boundaries of the 3D brain sub-representation, splitting a representation of infusate into infusate located within the 3D brain sub-representation and infusate outside the 3D brain sub-representation;
mapping the infusate located within the 3D brain sub-representation to a first visual indicator and the infusate located outside the 3D brain sub-representation to a second visual indicator;
generating a 3D infusate delivery representation based on the mapping, the 3D infusate delivery representation comprising a first 3D infusate delivery sub-representation of infusate located within the 3D brain sub-representation and a second 3D infusate delivery sub-representation of infusate located outside the 3D brain sub-representation; and
generating a visualization of the 3D infusate delivery representations and the 3D brain sub-representation.

2. The method of claim 1, wherein splitting the representation of infusate into infusate located within the 3D brain sub-representation and infusate outside the 3D brain sub-representation comprises:
detecting intersections of the representation of infusate with the boundaries of the 3D brain sub-representation; and
generating a bitmask volume of the infusate located within the 3D brain sub-representation and infusate outside the 3D brain sub-representation comprises,
wherein the mapping of the infusate located within the 3D brain sub-representation to a first visual indicator and the infusate located outside the 3D brain sub-representation to a second visual indicator is based on the bitmask volume.

3. The method of claim 2, wherein generating a bitmask volume of the infusate located within the 3D brain sub-representation and infusate outside the 3D brain sub-representation comprises:
assigning a first numerical value to the infusate located within the 3D brain sub-representation and a second numerical value to the infusate outside the 3D brain sub-representation comprises.

4. The method of claim 3, wherein mapping the infusate located within the 3D brain sub-representation to the first visual indicator and the infusate located outside the 3D brain sub-representation to the second visual indicator comprises:
accessing a visual indicator lookup table comprising associations of the first visual indicator to the first numerical value and the second visual indicator to the second numerical value; and
assigning the first visual indicator to the infusate located within the 3D brain sub-representation based on the first numerical value and the second visual indicator to the infusate outside the 3D brain sub-representation based on the second numerical value.

5. The method of claim 1, wherein generating the 3D infusate delivery representation based on the mapping comprises:
extracting first 3D meshed surfaces for the infusate located within the 3D brain sub-representation and second 3D meshed surfaces for infusate located outside of the 3D brain sub-representation, wherein the first 3D infusate delivery sub-representation comprises the first 3D meshed surfaces and the second 3D infusate delivery sub-representation comprises the second 3D meshed surfaces.

6. The method of claim 1, further comprising:
based on the 3D infusate delivery representation and the 3D brain sub-representation, estimating one or more of a volume of infusate located within the one of a target structure or region, a level of coverage for delivered infusate within the target structure or region, and a leakage volume of infusate located outside the target structure or region.

7. The method of claim 6, further comprising:
detecting intersections between the 3D infusate delivery representation and the 3D brain sub-representation; and
estimating one or more of:
the volume of infusate located within the one of a target structure or region by computing a volume of the first 3D infusate delivery sub-representation;
the level of coverage based on computing a ratio a volume of the first 3D infusate delivery sub-representation over a volume of the 3D brain sub-representation; and
the leakage volume by computing a volume of the second 3D infusate delivery sub-representation.

8. The method of claim 6, wherein the 3D brain sub-representation, the first 3D infusate delivery sub-representation, and the second 3D infusate delivery sub-representation each comprise a respective 3D mesh boundary surface.

9. The method of claim 1, further comprising:
obtaining the representation of infusate based on one of:
subtracting a first image of a patient's brain acquired after infusate has been delivered to the patient's brain from a second image of the patient's brain acquired before infusate has been delivered to the patient's brain; and
simulating a biophysical infusion model that simulates delivery of the infusate in the patient's brain.

10. A system comprising:
a memory storing instructions; and
a hardware processor coupled to the memory and configured to execute the instructions to:
extract boundaries of a 3D brain sub-representation by segmenting a patient-specific 3D brain representation, the patient-specific 3D brain sub-representation representing a target structure or region of a brain of a patient;
based on the boundaries of the 3D brain sub-representation, segment a representation of infusate into infusate located within the 3D brain sub-representation and infusate outside the 3D brain sub-representation;
map the infusate located within the 3D brain sub-representation to a first visual indicator and the infusate located outside the 3D brain sub-representation to a second visual indicator;
generate a 3D infusate delivery representation based on the mapping, the 3D infusate delivery representation comprising a first 3D infusate delivery sub-representation of infusate located within the 3D brain sub-representation and a second 3D infusate delivery sub-representation of infusate located outside the 3D brain sub-representation; and
generate a visualization of the 3D infusate delivery representations and the 3D brain sub-representation

11. The system of claim 10, wherein splitting the representation of infusate into infusate located within the 3D brain sub-representation and infusate outside the 3D brain sub-representation comprises:
detecting intersections of the representation of infusate with the boundaries of the 3D brain sub-representation; and
generating a bitmask volume of the infusate located within the 3D brain sub-representation and infusate outside the 3D brain sub-representation comprises,
wherein the mapping of the infusate located within the 3D brain sub-representation to a first visual indicator and the infusate located outside the 3D brain sub-representation to a second visual indicator is based on the bitmask volume.

12. The system of claim 11, wherein generating a bitmask volume of the infusate located within the 3D brain sub-representation and infusate outside the 3D brain sub-representation comprises:
assigning a first numerical value to the infusate located within the 3D brain sub-representation and a second numerical value to the infusate outside the 3D brain sub-representation comprises.

13. The system of claim 12, wherein mapping the infusate located within the 3D brain sub-representation to the first visual indicator and the infusate located outside the 3D brain sub-representation to the second visual indicator comprises:
accessing a visual indicator lookup table comprising associations of the first visual indicator to the first numerical value and the second visual indicator to the second numerical value; and
assigning the first visual indicator to the infusate located within the 3D brain sub-representation based on the first numerical value and the second visual indicator to the infusate outside the 3D brain sub-representation based on the second numerical value.

14. The system of claim 10, wherein generating the 3D infusate delivery representation based on the mapping comprises:
extracting first 3D meshed surfaces for the infusate located within the 3D brain sub-representation and second 3D meshed surfaces for infusate located outside of the 3D brain sub-representation,
wherein the first 3D infusate delivery sub-representation comprises the first 3D meshed surfaces and the second 3D infusate delivery sub-representation comprises the second 3D meshed surfaces.

15. The system of claim 10, wherein the hardware processor is further configured to execute the instructions to:
based on the 3D infusate delivery representation and the 3D brain sub-representation, estimate one or more of a volume of infusate located within the one of a target structure or region, a level of coverage for delivered infusate within the target structure or region, and a leakage volume of infusate located outside the target structure or region.

16. The system of claim 15, wherein the hardware processor is further configured to execute the instructions to:
detect intersections between the 3D infusate delivery representation and the 3D brain sub-representation; and
estimating one or more of:
the volume of infusate located within the one of a target structure or region by computing a volume of the first 3D infusate delivery sub-representation;
the level of coverage by computing a ratio a volume of the first 3D infusate delivery sub-representation over a volume of the 3D brain sub-representation; and
the leakage volume by computing a volume of the second 3D infusate delivery sub-representation.

17. The system of claim 15, wherein the 3D brain sub-representation, the first 3D infusate delivery sub-representation, and the second 3D infusate delivery sub-representation each comprise a respective 3D mesh boundary surface.

18. The system of claim 10, wherein the hardware processor is further configured to execute the instructions to:
obtain the representation of infusate based on one of:
subtracting a first image of a patient's brain acquired after infusate has been delivered to the patient's brain from a second image of the patient's brain acquired before infusate has been delivered to the patient's brain; and
simulating a biophysical infusion model that simulates delivery of the infusate in the patient's brain.

19. A non-transitory computer-readable storage medium including instructions that, when executed by at least one processor of a computing system, cause the computing system to perform a method comprising:
segment a representation of infusate provided to a 3D representation of a target brain structure of patient into a plurality of sub-representations of infusate;
map the plurality of sub-representations of infusate to a plurality of visual indicators;
extract 3D closed mesh boundaries for each of the plurality of sub-representations of infusate; and
execute a graphical user interface to generate a visualization of the extracted 3D closed mesh boundaries of the plurality of sub-representations combined with a target 3D closed mesh boundary of the 3D representation of the target brain structure of patient, wherein the extracted 3D closed mesh boundaries are generated based on the plurality of visual indicators mapped to respective sub-representations of infusate of the plurality of sub-representations of infusate.

20. The non-transitory computer-readable medium of claim 19, wherein the method further comprises:
extracting the target 3D closed mesh boundary of the 3D representation of the target brain structure,
wherein segmenting the representation of infusate is based on the combining the representation of infusate with the target 3D closed mesh boundary.
